(19) United States
(12) Patent Application Publication
(10) Pub. No.: US 2004/0167375 A1
(43) Pub. Date: Aug. 26, 2004

(54) Cardiac assist device with electroactive polymers

(76) Inventor: Lucien A. Couvillon JR., Concord, MA (US)

Correspondence Address: Joseph R. Kelly, WESTMAN CHAMPLIN & KELLY, International Centre - Suite 1600, 900 South Second Avenue, Minneapolis, MN, 55402-3319, US

(21) Appl. No.: 10/373,940

(22) Filed: Feb. 25, 2003

(51) Int. Cl. A61N 1/362

(52) U.S. Cl. 600/17

(57) ABSTRACT

The present invention is directed to a cardiac assist device for assisting with the function of a heart. The assist device includes a compressor positioned adjacent the epicardial wall of the heart. The compressor is driven by one or more electroactive polymer actuators. The pressure exerted against the heart improves heart function.

### BACKGROUND OF THE INVENTION

The present invention deals with a ventricular assist device. More particularly, the present invention deals with a device for direct mechanical assistance to the failing heart by the application of electroactive polymer actuators.

A number of different types of coronary disease and heart failure can require ventricular assist. One class of present ventricular assist devices (VADs) employ mechanical pumps to circulate blood through the vasculature. These pumps are typically plumbed between the apex of the left ventricle and the aortic arch (for LVADs), and provide mechanical assistance to a weak heart. These devices must be compatible with the blood, and inhibit thrombus formation, due to the intimate contact between the pump components and the blood.

Another class of ventricular assistance, direct mechanical ventricular assistance, includes squeezing the heart from the epicardial surface to assist the ejection of blood from the ventricles during systole. This form of ventricular assist does not require contact with blood or surgical entry into the cardiovascular system. It has been expressed in several embodiments over the years. The first involves an approach which is drastically different from the mechanical pumps approach discussed above. The approach uses a muscle in the patient's back. The muscle is detached and wrapped around the epicardium of the heart. The muscle is then trained to contract in synchrony with the ECG pulse, or other pulse (which may be generated by a pacemaker). Since the back muscle does not contact blood, many of the issues faced by conventional LVADs are avoided. However, this approach also suffers from disadvantages, because operation of the muscle tissues is poorly understood and largely uncontrolled.

A number of other methods are also taught by prior references. Some such references disclose balloons or bellows which squeeze on the exterior surface of the heart in synchrony with the ECG signal. U.S. Pat. No. 3,455,298 to Anstadt discloses an air pressure source which is used to inflate a cup-shaped balloon chamber about a portion of the external surface of the heart, in order to provide a squeezing pressure on the heart.

Other references disclose similar items which are inflated using fluid inflation devices. Still other references disclose mechanical means which apply pressure radially inwardly on the epicardial surface of the heart. For instance, U.S. Pat. No. 4,621,617 to Sharma discloses an electromechanical mechanism for applying external pressure to the heart.

Similarly, in order to address heart failure (and sometimes for organ preservation) in accordance with other prior approaches, a patient's heart is placed within a cup-shaped device that applies pulsatile force to express blood from the ventricles. This is done in order to keep the patient alive, or in order to keep the organ viable for transplantation. Some such systems use pneumatic actuators which are bulky, inefficient, noisy, expensive, slow, and can be very difficult to control.

### SUMMARY OF THE INVENTION

The present invention is directed to a cardiac assist device for assisting with the function of a heart. The assist device includes a compressor positioned adjacent the epicardial wall of the heart. The compressor is driven by one or more electroactive polymer actuators. The pressure exerted against the heart improves heart function.

### BRIEF DESCRIPTION OF THE DRAWINGS

FIG. 1 illustrates a partial sectional view of a human heart and its associated proximate vascular system.

FIG. 2 is a diagrammatic illustration of a cardiac assist device in accordance with one embodiment of the present invention.

FIG. 3 is a diagrammatic view of the system shown in FIG. 2 placed in compressive relation to a heart.

### DETAILED DESCRIPTION OF THE ILLUSTRATIVE EMBODIMENTS

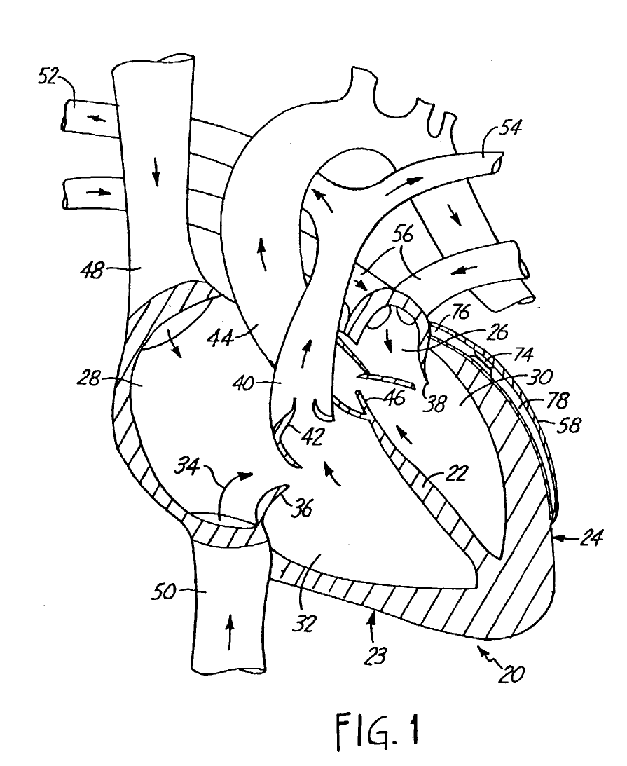

FIG. 1 illustrates a partially sectioned view of a human heart 20, and its associated vasculature. The heart 20 is subdivided by muscular septum 22 into two lateral halves, which are named respectively right 23 and left 24. A transverse constriction subdivides each half of the heart into two cavities, or chambers. The upper chambers consist of the left and right atria 26, 28 which collect blood. The lower chambers consist of the left and right ventricles 30, 32 which pump blood. The arrows 34 indicate the direction of blood flow through the heart. The chambers are defined by the epicardial wall of the heart.

The right atrium 28 communicates with the right ventricle 32 by the tricuspid valve 36. The left atrium 26 communicates with the left ventricle 30 by the mitral valve 38. The right ventricle 32 empties into the pulmonary artery 40 by way of the pulmonary valve 42. The left ventricle 30 empties into the aorta 44 by way of the aortic valve 46.

The circulation of the heart 20 consists of two components. First is the functional circulation of the heart 20, i.e., the blood flow through the heart 20 from which blood is pumped to the lungs and the body in general. Second is the coronary circulation, i.e., the blood supply to the structures and muscles of the heart 20 itself.

The functional circulation of the heart 20 pumps blood to the body in general, i.e., the systematic circulation, and to the lungs for oxygenation, i.e., the pulmonic and pulmonary circulation. The left side of the heart 24 supplies the systemic circulation. The right side 23 of the heart supplies the lungs with blood for oxygenation. Deoxygenated blood from the systematic circulation is returned to the heart 20 and is supplied to the right atrium 28 by the superior and inferior venae cavae 48, 50. The heart 20 pumps the deoxygenated blood into the lungs for oxygenation by way of the main pulmonary artery 40. The main pulmonary artery 40 separates into the right and left pulmonary arteries, 52, 54 which circulate to the right and left lungs, respectively. Oxygenated blood returns to the heart 20 at the left atrium 26 via four pulmonary veins 56 (of which two are shown). The blood then flows to the left ventricle 30 where it is pumped into the aorta 44, which supplies the body with oxygenated blood.

The functional circulation, however, does not supply blood to the heart muscle or structures. Therefore, functional circulation does not supply oxygen or nutrients to the heart 20 itself. The actual blood supply to the heart structure, i.e., the oxygen and nutrient supply, is provided by the coronary circulation of the heart, consisting of coronary arteries, indicated generally at 58, and cardiac veins. Coronary artery 58 resides closely proximate the endocardial wall of heart 24. The coronary artery 58 includes a proximal arterial bed 76 and a distal arterial bed 78 downstream from the proximal bed 76.

In order to assist the heart, one embodiment of the present invention provides a compressor disposed about a periphery of the heart. The compressor is located closely proximate the epicardial surface of the heart and is driven by the movement of electroactive polymer actuators in order to assist the heart.

Prior to discussing the present invention in greater detail a brief description of one illustrative embodiment of the actuators used in accordance with the present invention will be undertaken. Electroactive polymer actuators typically include an active member, a counter-electrode and an electrolyte containing region disposed between the active member and the counter-electrode. In some embodiments, a substrate is also provided, and the active member, the counter-electrode and the electrolyte-containing region are disposed over the substrate layer. Some examples of electroactive polymers that can be used as the electroactive polymer actuators of the present invention include polyaniline, polypyrrole, polysulfone, polyacetylene.

Actuators formed of these types of electroactive polymers are typically small in size, exhibit large forces and strains, are low cost and are relatively easy to integrate into a cardiac assist device. These polymers are members of the family of plastics referred to as “conducting polymers” which are characterized by their ability to change shape in response to electrical simulation. They typically structurally feature a conjugated backbone and have the ability to increase electrical conductivity under oxidation or reduction. These materials are typically not good conductors in their pure form. However, upon oxidation or reduction of the polymer, conductivity is increased. The oxidation or reduction leads to a charge imbalance that, in turn, results in a flow of ions into the material in order to balance charge. These ions or dopants, enter the polymer from an ionically conductive electrolyte medium that is coupled to the polymer surface. The electrolyte may be, for example, a gel, a solid, or a liquid. If ions are already present in the polymer when it is oxidized or reduced, they may exit the polymer.

It is well known that dimensional changes may be effectuated in certain conducting polymers by the mass transfer of ions into or out of the polymer. For example, in some conducting polymers, the expansion is due to ion insertion between changes, wherein as in others inter-chain repulsion is the dominant effect. Thus, the mass transfer of ions into and out of the material leads to an expansion or contraction of the polymer.

Currently, linear and volumetric dimensional changes on the order of 25 percent are possible. The stress arising from the dimensional change can be on the order of three MPa, far exceeding that exhibited by smooth muscle cells, thereby allowing substantial forces to be exerted by actuators having very small cross-sections. These characteristics are favorable for construction of a cardiac assist device in accordance with the present invention.

Additional information regarding the construction of actuators, their design considerations and the materials and components that maybe deployed therein can be found, for example, in U.S. Pat. No. 6,249,076 assigned to Massachusetts Institute of Technology, and in proceedings of the SPIE Vol. 4329 (2001) entitled Smart Structures and Materials 2001: Electroactive Polymer and Actuator Devices (see in particular, Madden et al., Polypyrrole actuators: Modeling and Performance at pp. 72-83), and in U.S. patent application Ser. No. 10/262,829 entitled Thrombolysis Catheter assigned to the same assignee as the present invention.

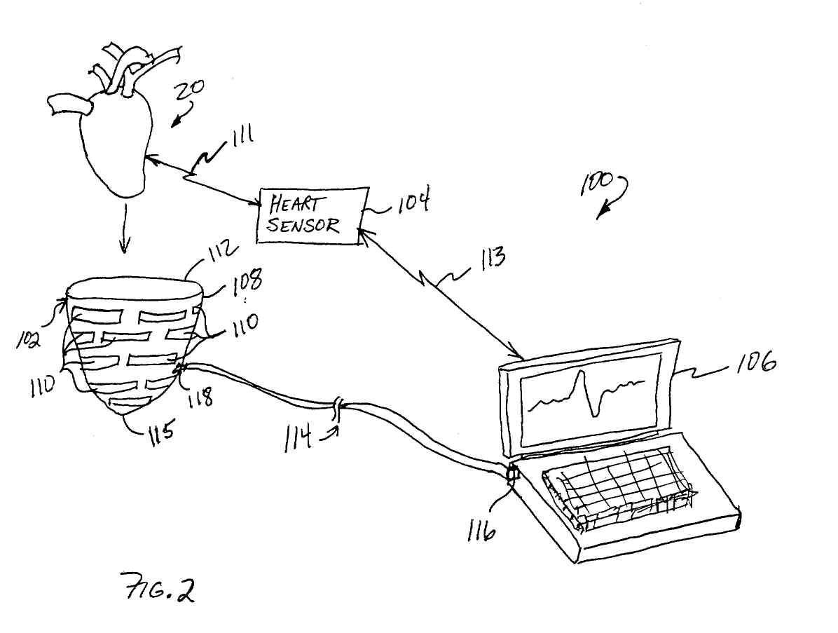

FIG. 2 is a diagrammatic representation of a cardiac assist system 100 in accordance with one embodiment of the present invention. Cardiac assist system 100 shows heart 20, compressor 102, heart sensor 104 and computing device 106. Compressor 102 can illustratively be formed of a sock or cup-shaped receiver 108 with a plurality of electroactive polymer actuators 110 disposed thereon. Receiver 108 includes a first open end 112 and a second end 115. In the embodiment shown in FIG. 2, open end 112 is sized to receive heart 20 therein and end 115 is closed to securely receive the apex of heart 20. However, it should be noted that receiver 108 can be open at both ends or be of a different shape, so long as it closely conforms to the epicardiam of heart 20.

In addition, receiver 108 is illustratively formed of a generally flexible material which can move under the influence of actuators 110 to exert pressure on heart 20 and then to relax to allow heart 20 to expand. Receiver 108 can thus be formed of any suitable material, such as a flexible polymer, a flexible mesh or woven fabric.

Heart sensor 104 can illustratively be a heart rate monitor, or any other type of sensor which can be used to sense the sinus rhythm of heart 20. Of course, where system 100 is deployed simply to preserve organs for transplantation, heart sensor 104 is optional, and is replaced by a simple pulse generator. If heart 20 has stopped beating, it can be pulsed using system 100 without reference to, or feedback from, its natural sinus rhythm.

In any case, when sensor 104 is used, it senses desired characteristics of heart 20 through a connection 111 which can simply be a conductive contact-type connection, or other known connection, including traditional body-surface EKG electrodes. Sensor 104 is also illustratively connected to computing device 106 through a suitable connection 113. Connection 113 can be a hard wired connection, a wireless connection (such as one using infrared or other electromagnetic radiation) or any other desired connection.

Computing device 106 can be any of a wide variety of computing devices. While computing device 106 is generally illustrated in FIG. 2 as a laptop computer, it can be a desktop computer, a personal digital assistant (PDA), a palmtop or handheld computer, even a mobile phone or other computing device, or a dedicated special-purpose electronic control device. In addition, computing device 106 can be stand-alone, part of a network or simply a terminal which is connected to a server or another remote computing device. The network (if used) can include a local area network (LAN), a wide area network (WAN), wireless link, or any other suitable configuration.

In any case, computing device 106 illustratively includes a communication interface, or power interface, for providing signals to electroactive polymer actuators 110 through a link 114. The power interface can be a transcutaneous transformer of the type commonly used with implantable artificial heart or LVAD systems.

Connection 114 is shown as a cable that has a first connector 116 connected to the communication or power electronics in computing device 106 and a second connector 118 which is connected to provide signals to actuators 110. It should also be noted, however, that connection 114 can also be a different type of connection, such as a wireless connection, which provides the desired signals to actuators 110 using electromagnetic energy, or any other desired type of link.

Actuators 110 can be applied to receiver 108 by weaving them into receiver 108, depositing them on receiver 108, mechanically attaching them to receiver 108 (such as with sutures or adhesive) or by any other method of disposing them on receiver 108 such that, when they contract, they drive compression of compressor 102.

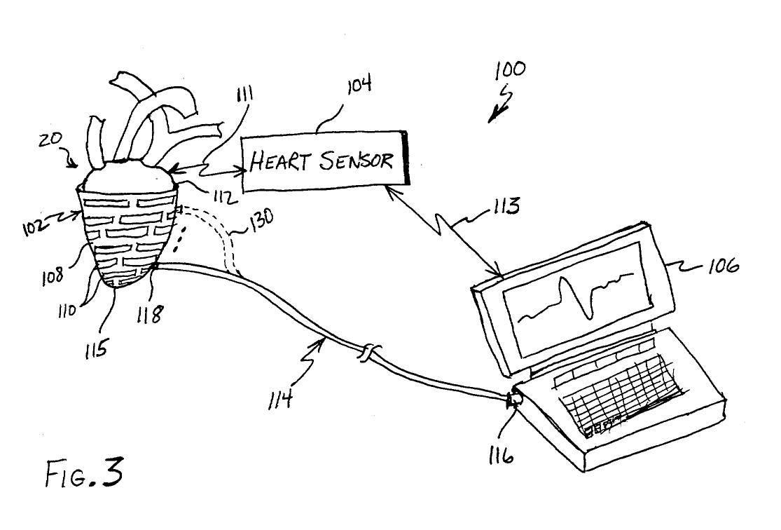

FIG. 3 shows system 100 in which heart 20 has been placed inside compressor 102. During operation, the patient's chest can be opened for resuscitation. In that embodiment, heart 20 of the patient is placed in compressor 102. Compressor 102 illustratively snugly engages the exterior periphery of heart 20. Sensor 104 senses the sinus rhythm of heart 20 and provides a signal indicative of that rhythm to computing device 106. Based on the sinus rhythm of heart 20, computing device 106 provides signals over link 114 to the actuators 110. In one embodiment, the signals cause the actuators to contract according to a timing that is synchronous with the desired sinus rhythm of heart 20. When actuators 110 contract, they cause compressor 102 to exert a compressive force on heart 20 thereby assisting the compressive portion of the heart function.

In order to reduce the likelihood that heart 20 will slip out of compressor 102 upon compression, heart 20 can be disconnectably secured within compressor 102. This can be done in any of a variety of ways, such as using a small number of sutures, a suitable clamping device, or any type of retractable or removable connection mechanism.

It should be noted that different pulsation techniques can be implemented. For example, the signals provided from computing device 106 over connection 114 can be provided to all of actuators 110 at once, thus pulsing the whole heart 20 at once. Alternatively, however, a plurality of connective ends 130 can be provided that include conductors carrying additional signals provided by computing device 106. In that embodiment, computing device 106 can provide these signals to more closely mimic the natural “wringing”, propagating-pulsing action of heart 20. Therefore, for instance, computing device 106 can provide signals which cause the actuators 110 closer to the apex of heart 20 to contract first and those further from the apex to contract later. Any number of optional additional connections 130 can be provided so long as the appropriate signals are provided from computing device 106.

It should also be noted that, in another embodiment, compressor 102 is implantable and connection link 114 is wireless. In that embodiment, computing device 106 simply needs to be able to provide sufficient energy over wireless link 114 to initiate contraction of actuators 110. Similarly, additional power circuitry can be deployed on compressor 102 to amplify these signals provided by computing device 106 over wireless link 114 in order to cause contraction of actuators 110.

Also, while other actuators are alternatives to EAP, such as piezoelectric or shape memory actuators, they may be less efficient, larger and more expensive than electroactive polymers. The small size and efficiency of electroactive polymers provide great flexibility in the placement and control of the pumping assist forces. The low activation voltage and high efficiency of the electroactive polymers allow the use of simple, small drive and monitoring circuits, such as those found in conventional personal computer card interfaces. Similarly, the electroactive polymers can provide better fit to the heart 20, better application of pressure, a small profile, and better control of pulsation forces.

Although the present invention has been described with reference to preferred embodiments, workers skilled in the art will recognize that changes may be made in form and detail without departing from the spirit and scope of the invention.

What is claimed is:

1. A system for assisting a heart, comprising:
a compressor; and
an electroactive polymer (EAP) actuator coupled to the compressor.

2. The system of claim 1 and further comprising:
an electrical driver operably connected to the EAP actuator.

3. The system of claim 2 and further comprising:
a heart sensor.

4. The system of claim 3 wherein the heart sensor senses heart contraction and provides a heart rate signal indicative of heart rate.

5. The system of claim 4 wherein the electrical driver includes a computing device receiving the heart rate signal and providing an actuator driver signal to actuate the EAP actuator.

6. The system of claim 1 wherein the compressor comprises:
a receiver having an inner periphery defining an opening sized to receive the heart.

7. The system of claim 6 wherein the EAP actuator is connected to the receiver.

8. The system of claim 7 wherein the EAP actuator is connected to the receiver with adhesive.

9. The system of claim 7 wherein the EAP actuator is connected to the receiver with sutures.

10. The system of claim 7 wherein the EAP actuator is woven into the receiver.

11. The system of claim 7 wherein the receiver comprises:
a mesh.

12. The system of claim 7 wherein the receiver comprises:
a woven sock.

13. The system of claim 1 wherein the receiver comprises:
a bag of flexible material.

14. The system of claim 2 wherein the EAP actuator comprises:
a plurality of EAP actuator members disposed about a periphery of the compressor.

15. The system of claim 14 wherein the electrical driver provides a plurality of driving signals driving actuation of different ones of the plurality of EAP actuator members at different times.

16. A system for compressing a body organ, comprising:
a flexible receiver sized to receive the body organ therein; and
an electroactive polymer (EAP) actuator connected to the receiver.

17. The system of claim 16 wherein the EAP actuator comprises:
a plurality of EAP actuator members disposed about a periphery of the receiver.

18. The system of claim 17 and further comprising:
a driver providing a driving signal to the plurality of EAP actuator members to drive physical movement of the EAP actuator members.

19. The system of claim 18 and further comprising:
a sensor, coupled to the driver and sensing natural movement of the body organ.

20. The system of claim 19 wherein the driver provides the driving signal based on sensed natural movement of the body organ.

21. A method of compressing a heart, comprising:
placing the heart in a flexible receiver having an electoactive polymer (EAP) actuator disposed thereon; and
providing an electrical driving signal to the EAP actuator to drive actuation thereof.

22. The method of claim 21 and further comprising:
sensing natural heart function.

23. The method of claim 22 wherein providing an electrical driving signal comprises:
providing the electrical driving signal based on the sensed natural heart function.

24. The method of claim 21 wherein the EAP actuator comprises a plurality of EAP actuator members and wherein providing an electrical drive signal comprises:
providing the electrical drive signal to the plurality of EAP actuator members.

25. The method of claim 24 wherein providing the electrical drive signal comprises:
providing the electrical drive signal such that the plurality of EAP actuator members actuate at different times.
